(19) United States
(12) Patent Application Publication
(10) Pub. No.: US 2025/0303111 A1
(43) Pub. Date: Oct. 2, 2025

(54) BENDABLE GUIDEWIRE LUMEN

(71) Applicants: Bradley Howard STRAUSS, Toronto, Ontario (CA); Gilad CIBULSKI, Kadima (IL)

(72) Inventors: Bradley Howard STRAUSS, Toronto, Ontario (CA); Gilad CIBULSKI, Kadima (IL)

(21) Appl. No.: 18/865,396

(22) PCT Filed: May 11, 2023
(86) PCT No.: PCT/IL2023/050492

§ 371 Date: Nov. 13, 2024

Related U.S. Application Data
(60) Provisional application No. 63/341,431, filed on May 13, 2022.

(51) Int. Cl. A61M 25/00; A61M 25/01

(52) U.S. Cl. CPC A61M 25/0021; A61M 25/0052; A61M 25/007; A61M 25/0108; A61M 25/0147; A61M 2025/0004; A61M 2025/0042; A61M 2025/015; A61M 2205/0216; A61M 2205/32

(57) ABSTRACT

An aspect of some embodiments of the invention relate to a redirection microcatheter, comprising an external tubular body comprising a first proximal end and a first distal end; said first distal end comprising a distal end opening and at least one side opening; an internal tubular body sized and shaped to be inserted within said external tubular body; said internal tubular body comprising a second proximal end and a second distal end; said second distal end located adjacent to said at least one side opening; at least one actuator for translating said second distal end of said internal tubular body from an un-actuated position where said second distal end of said internal tubular body faces said distal opening to an actuated position where said second distal end of said internal tubular body faces said one or more side openings in said external tubular body.

### RELATED APPLICATION/S

This application claims the benefit of priority of U.S. Provisional Patent Application No. 63/341,431 filed on 13 May 2022, the contents of which are incorporated herein by reference in their entirety.

### FIELD AND BACKGROUND OF THE INVENTION

The present invention, in some embodiments thereof, relates to catheters with dedicated guidewire lumens and, more particularly, but not exclusively, to catheters with bendable internal lumens.

Additional background art includes U.S. Pat. No. 6,217,527B1 disclosing methods and apparatus for crossing totally to substantially occluded blood vessels by passing a redirectable wire such as a guidewire from a relatively proximal point past the occlusion within a subintimal space formed between the intimal layer and the adventitial layer of a blood vessel wall. The wire may be advanced to a point distal to the occlusion, and thereafter deflected back into the blood vessel lumen, typically using a deflecting catheter, which is advanced over the guidewire after it has been positioned within the subintimal space. The deflecting catheter may include a flapper valve assembly or preformed actuator wire for redirecting the guidewire. After the guidewire is returned to the blood vessel lumen, the deflecting catheter may be withdrawn, and the guidewire may be available for introduction of other interventional and diagnostic catheters for performing procedures such as stenting.

U.S. Patent Application Publication No. US20200282187A1 disclosing an obturator comprising a hollow distal end portion, the distal end portion comprising a distal end and a side hole located proximally of the distal end, the side hole being for receipt of a guidewire and to direct the guidewire laterally from the obturator.

U.S. Pat. No. 9,233,224B1 disclosing a medical device and method for accessing a side branch in an artery. The device includes a catheter having a sidewall, an internal lumen, and a side port formed through the sidewall. A perforating guide wire has a proximal portion within the internal lumen and a distal portion arranged to be movable out of the side port. The guide wire can be delivered through the side port to a side branch artery when the catheter is deployed to a location with the side port aligned with the side branch artery. In another embodiment, the catheter has inner and outer telescoping tubes with offset exit ports formed therein. The telescoping tubes can be used to change the degree of deflection of the perforating guide wire by changing the relative positions of the offset exit ports.

U.S. Patent Application Publication No. US20020055733A1 disclosing a catheter assembly and method for delivering guide wires to vessels. The catheter assembly of the present invention has the feature of containing two guide wire lumens in a single catheter to prevent wire wrapping and crossing of the wires. A torquing member is provided to assist in rotation of the catheter. The assembly allows for the delivery of a wire into a side branch vessel that is at a steep angle with respect to a main vessel. The assembly also provides for the delivery of two guide wires to a bifurcation.

U.S. Patent Application Publication No. US20210236774A1 disclosing a catheter that allows selective direction of a surgical tool into multiple blood vessels of a patient. The catheter includes a catheter body that has a main exit port and a side exit port. An internal balloon is provided within the catheter body. The internal balloon is inflatable, and can be located at or near a distal portion of the side exit port. When deflated, the internal balloon allows the surgical tool to advance past the side exit port and out the main exit port. When inflated, the internal balloon directs the surgical tool to advance out the side exit port instead of the main exit port.

U.S. Patent Application Publication No. US20190091438A1 disclosing a catheter with a lumen and comprising side port(s) through the elongate body of the catheter at predetermined locations. The side port(s) may be positioned at anatomical locations of interest and a fluid, e.g., a medicament, infused down the catheter's lumen, the fluid flowing out of the side port(s) to treat the anatomical location. Alternative embodiments may include a translatable inner sheath that may cover some of the side port(s) and may also include an aperture that may align with at least one of the side port(s). The side ports may be opened and closed by means of an actuator such as a push/pull wire or an electrically response shape memory material. A selective filter may be used to cover the side ports, whereby only fluids of certain viscosities and/or molecular size may pass through the filter and side port.

U.S. Patent No. 9,610,438B2 disclosing a delivery catheter, including a catheter body, a side port, a first electrode, and a second electrode. The catheter body may comprise a proximal end, a distal end, and a perimeter surface. The catheter body defines a delivery lumen extending longitudinally within the catheter body. The side port is defined in the perimeter surface of the catheter body proximate the distal end and in communication with the delivery lumen. The electrodes may be adjacent to and spaced from the side port. Techniques for using the delivery catheter to identify a desired lead implantation location, e.g., via the electrodes, and implant a medical lead or other implantable element at the desired location through the delivery lumen and side port are also described.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A redirection microcatheter, comprising:
a. an external tubular body comprising a first proximal end and a first distal end; said first distal end comprising a distal end opening and at least one side opening;
b. an internal tubular body sized and shaped to be inserted within said external tubular body; said internal tubular body comprising a second proximal end and a second distal end; said second distal end located adjacent to said at least one side opening;
c. at least one actuator for translating said second distal end of said internal tubular body from an un-actuated position where said second distal end of said internal tubular body faces said distal opening to an actuated position where said second distal end of said internal tubular body faces said one or more side openings in said external tubular body.

Example 2. The redirection catheter according to example 1, wherein said actuator comprises at least one holder coupled to a location adjacent to said second distal end of said internal tubular body, and at least one wire coupled to said at least one holder.

Example 3. The redirection catheter according to example 1 or example 2, wherein said translating comprises bending said second distal end of said internal tubular body an angle of from about 1 degree to about 150 degrees.

Example 4. The redirection catheter according to any one of examples 1-3, wherein said translating comprises bending said second distal end of said internal tubular body an angle of about 90 degrees.

Example 5. The redirection catheter according to any one of examples 1-4, w herein said at least one side opening is at a distance of from about 0.5 mm to about 10 mm from said distal opening.

Example 6. The redirection catheter according to any one of examples 1-5, wherein said distal opening comprises a size of from about 0.015″ to about 0.1.″

Example 7. The redirection catheter according to any one of examples 1-6, wherein said at least one side opening comprises a size of from about 0.015″ to about 0.1.″

Example 8. The redirection catheter according to any one of examples 1-7, wherein said external tubular body is made of one or more of nylon, Pebax, polyorithen, metal, PTFE and any combination thereof.

Example 9. The redirection catheter according to any one of examples 1-8, wherein said external tubular body is a braided/coiled shaft.

Example 10. The redirection catheter according to any one of examples 1-9, wherein said external tubular body comprises a length of from about 90 cm to about 180 cm.

Example 11. The redirection catheter according to any one of examples 1-10, wherein said external tubular body comprises a size that fits through a 5 F or 6 F sheath.

Example 12. The redirection catheter according to any one of examples 1-10, wherein said external tubular body comprises a reinforced area located adjacent and/or around to said one or more side openings.

Example 13. The redirection catheter according to example 12, wherein said reinforced area comprises one or more metallic elements configured to increase a level of rigidity in said reinforced area.

Example 14. The redirection catheter according to any one of examples 1-13, wherein said internal tubular body is made of a flexible material.

Example 15. The redirection catheter according to any one of examples 1-14, wherein at least a part of said internal tubular body is made of a flexible material.

Example 16. The redirection catheter according to any one of examples 1-15, wherein at least a distal part of the internal tubular body is made of a flexible material.

Example 17. The redirection catheter according to any one of examples 1-16, wherein said flexible part of said internal tubular body comprises a hardness of from about 20 D Shore to about 60 D Shore.

Example 18. The redirection catheter according to any one of examples 1-17, wherein said internal tubular body is made of one or more of nylon, Pebax, polyorithen, metal, PTFE and any combination thereof.

Example 19. The redirection catheter according to any one of examples 1-18, wherein said external tubular body is a braided/coiled shaft.

Example 20. The redirection catheter according to any one of examples 1-19, wherein said internal tubular body comprises a length of from about 100 mm to about 1800 mm.

Example 21. The redirection catheter according to any one of examples 1-20, wherein said internal tubular body comprises a length of about of that of said external tubular body.

Example 22. The redirection catheter according to any one of examples 1-20, wherein said second distal end extends until a distance of from about 0.1 cm to about 1.5 cm from said first distal end.

Example 23. The redirection catheter according to any one of examples 1-21, wherein said internal tubular body is configured to be extracted from said external tubular body thereby allowing insertion of other instruments within said external tubular body.

Example 24. The redirection catheter according to any one of examples 1-23, wherein said internal tubular body is configured to allow insertion of other instruments within itself without the need to be extracted from said external tubular body.

Example 25. The redirection catheter according to any one of examples 1-26, wherein said at least one actuator is made of one or more of stainless steel, nitinol, cobalt chromium, nylon, peek and Dynema.

Example 26. The redirection catheter according to any one of examples 1-25, wherein said at least one holder is a ring.

Example 27. The redirection catheter according to any one of examples 1-26, wherein said at least one holder is positioned on said flexible part of said internal tubular body.

Example 28. The redirection catheter according to any one of examples 1-27, wherein said at least one holder is positioned at a distance of from about 1 mm to about 20 mm from said second distal end.

Example 29. The redirection catheter according to any one of examples 1-28, wherein said at least one holder is arranged around said internal tubular body.

Example 30. The redirection catheter according to any one of examples 1-28, wherein said at least one holder is a groove on a surface of said internal tubular body.

Example 31. The redirection catheter according to any one of examples 1-29, wherein said at least one holder is made of one or more of stainless steel, nitinol, cobalt chromium, nylon, peek, platinum iridium and gold. Example 32. The redirection catheter according to any one of examples 1-31, wherein said internal tubular body is sized and shaped to allow the insertion of at least one guidewire within.

Example 33. The redirection catheter according to any one of examples 1-32, wherein said external tubular body comprises a first internal lumen in which said at least one actuator extends.

Example 34. The redirection catheter according to any one of examples 1-33, wherein said internal tubular body comprises a second internal lumen in which said at least one actuator extends.

Example 35. The redirection catheter according to any one of examples 1-34, wherein said internal tubular body comprises a third internal lumen in which said at least one guidewire is inserted.

Example 36. The redirection catheter according to any one of examples 1-35, further comprising at least one handle located proximally in said redirection catheter configured to receive one or more of said first proximal end of said external tubular body, said second proximal end of said internal tubular body and a third proximal end of said actuator.

Example 37. The redirection catheter according to any one of examples 1-36, further comprising at least one marking configured to indicate a direction to which said one or more side openings are facing.

Example 38. The redirection catheter according to example 37, wherein said at least one marking is a radiopaque marking.

Example 39. The redirection catheter according to example 37, wherein said at least one marking is located in one or more of the following locations: on the surface of said external tubular body, on the surface of said first distal end of said external tubular body, adjacent to said one or more side openings, on said at least one actuator and on said at least one holder.

Example 40. The redirection catheter according to any one of examples 1-39, further comprising a stabilizer configured to avoid bending of said first distal end when said at least one actuator is being actuated. Example 41. The redirection catheter according to example 40, wherein said stabilizer extends from said first proximal end to said first distal end. Example 42. The redirection catheter according to example 40, wherein said stabilizer extends within a lumen of said external tubular body.

Example 43. The redirection catheter according to example 40, wherein said stabilizer extends between said external tubular body and said internal tubular body.

Example 44. The redirection catheter according to example 40, wherein said stabilizer extends within a lumen of said external tubular body but outside of said internal tubular body.

Example 45. The redirection catheter according to example 40, wherein said stabilizer is configured to extend beyond said distal end opening when actuated.

Example 46. A method of actuating a redirection catheter according to example 1 while using a guidewire, the method comprising:
a. retracting said guidewire from said distal opening of said redirection catheter;
b. actuating said internal tubular body thereby translating said second distal end of said internal tubular body from an un-actuated position where said second distal end of said internal tubular body faces said distal opening to an actuated position where said second distal end of said internal tubular body faces said one or more side openings in said external tubular body;
c. moving distally said guidewire to cause said guidewire to exit from said at least one side opening in said redirection catheter.

Example 47. The method according to example 46, further comprising inserting said redirection catheter into a patient.

Example 48. The method according to example 46 or examples 47, further comprising directing said redirection catheter towards a desired location using said guidewire to assist with said directing.

Example 49. The method according to any one of examples 46-48, wherein said inserting further comprises inserting said guidewire into said redirection catheter.

Example 50. The method according to any one of examples 46-49, wherein said redirection catheter already comprises said guidewire inserted therein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIGS. 1a-1b are schematic representations of an exemplary redirection microcatheter, according to some embodiments of the invention;

FIGS. 1c-1e are schematic representations of an exemplary redirection microcatheter comprising a reinforced area, according to some embodiments of the invention;

FIGS. 2a-2b are schematic representations of an exemplary redirection microcatheter with a guidewire, according to some embodiments of the invention;

FIGS. 3a-3b are exemplary multi-lumen redirection catheters, according to some embodiments of the invention;

FIGS. 4a-4b are exemplary multi-lumen redirection catheters, according to some embodiments of the invention;

FIG. 5 is a flowchart of an exemplary method, according to some embodiments of the invention;

FIGS. 6a-6b are schematic representations of an exemplary optional stabilizer, according to some embodiments of the invention;

FIG. 7 is a flowchart of an exemplary method using an exemplary stabilizer, according to some embodiments of the invention.

### DESCRIPTION OF SPECIFIC EMBODIMENTS OF THE INVENTION

The present invention, in some embodiments thereof, relates to catheters with dedicated guidewire lumens and, more particularly, but not exclusively, to catheters with bendable internal lumens.

### Overview

An aspect of some embodiments of the invention relates to a redirection catheter configured to allow one or more instruments to exit the catheter from one or more side openings. In some embodiments, there are two or more side openings and the user can choose which side opening to use. In some embodiments, the redirection catheter comprises an internal tube located inside the redirection catheter configured to be directed towards the one or more side openings while the redirection catheter does not change direction. In some embodiments, the one or more side openings are big enough to allow the passage of one or more of a guidewire, a balloon catheter, a stent catheter, intervention instruments and drug releasing devices. In some embodiments, the internal tube can be directed to an angle from about 1 degree to about 150 degrees, preferably about 90 degrees, in relation to the longitudinal axis of the redirection catheter. In some embodiments, the redirection catheter is reinforced at the area where the internal tube is redirected. In some embodiments, the reinforcement are achieved by the addition of metallic inserts in the external tube. In some embodiments, the reinforcements are added over the entire area comprising the side opening. In some embodiments, the reinforcements are added at selected location in the vicinity or adjacent to the side opening. In some embodiments, the reinforcements avoid unwanted bending of the external tube at the location of the side opening. In some embodiments, the redirection catheter comprises a stabilizer. In some embodiments, the stabilizer is configured to avoid unwanted bending of the distal end of the external tube when the internal tube is being bent to allow for the redirection of the guidewire through the side opening. In some embodiments, the stabilizer is a wire extending from the proximal end of the external tube to the distal end of the external tube, within a lumen of the external tube. In some embodiments, the stabilizer is configured to move forward and backwards. In some embodiments, forward movement of the stabilizer allow a distal end of the stabilizer to exit through a distal opening in the external tube. In some embodiments, the stabilizer is stiff enough to stop the distal end of the external tube from bending when the internal tube in being bent.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

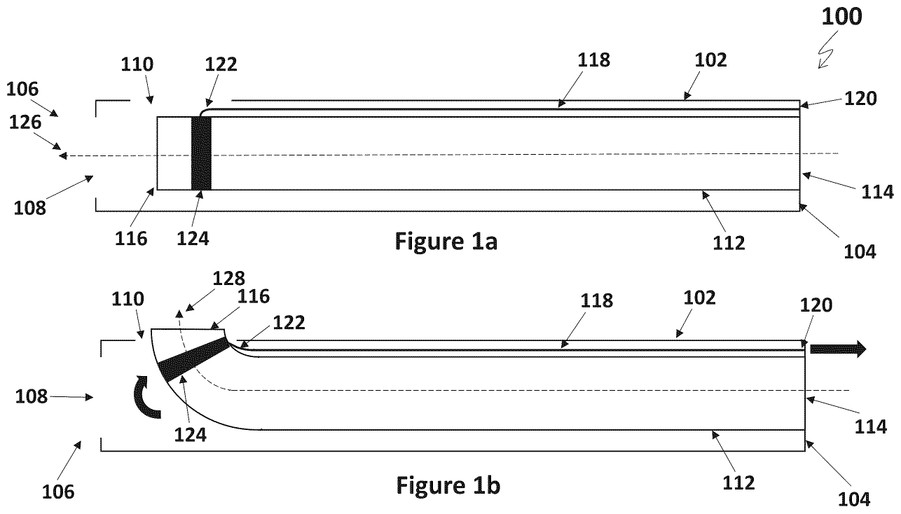

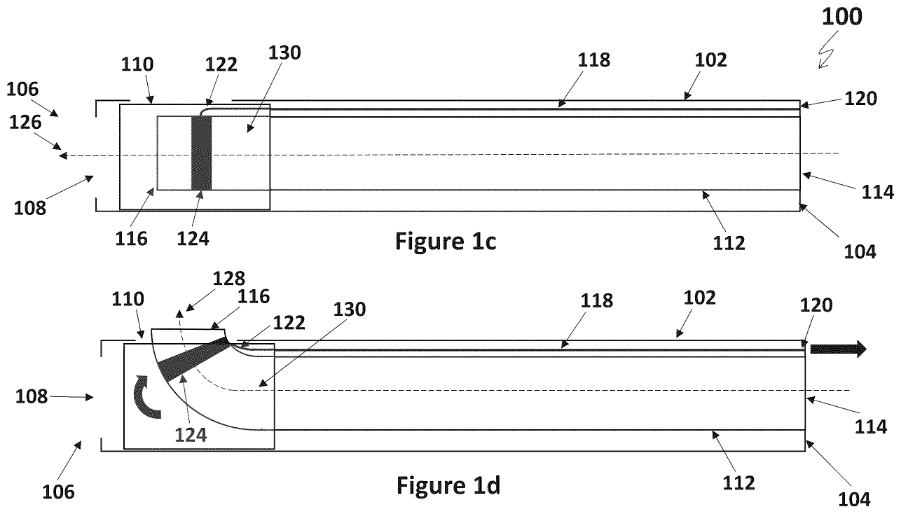

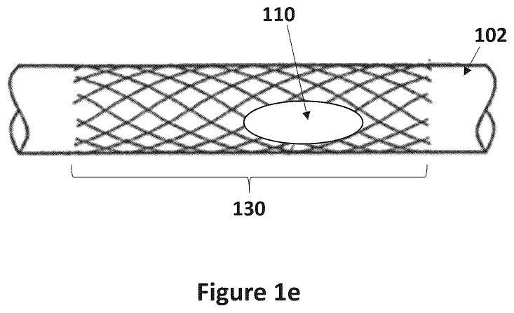

Referring now to FIGS. 1a-1b, showing schematic representations of an exemplary redirection microcatheter 100, according to some embodiments of the invention. In some embodiments, the redirection microcatheter 100 comprises an external tubular body 102 comprising a proximal end 104 and a distal end 106. In some embodiments, the proximal end is connected to an actuation handle (not shown). In some embodiments, the external tubular body 102 comprises a distal opening 108, located at the distal end 106 of the external tubular body 102, and at least one side opening 110 (referred herein after just as “side opening 110”—while mentioned in singular, it should be understood that the scope of the invention also includes more than one side opening) located adjacent and proximally to the distal end 106 of the external tubular body 102. In some embodiments, the external tubular body 102 comprises one side opening 110. In some embodiments, the external tubular body 102 comprises two side openings 110. In some embodiments, the external tubular body 102 comprises three or more side openings 110. In some embodiments, optionally, the distal opening 108 and/or the one or more side openings 110 are big enough to allow the passage of one or more of a guidewire (either a 0.035 mm guidewire or 0.018 mm guidewire or 0.014 mm guidewire), an internal tubular body (see below) a balloon catheter, a stent catheter and a microcatheter.

In some embodiments, the distal opening 108 comprises a size (or diameter) of from about 0.015″ to about 0.1″. For example, 0.015″, 0.02″, 0.036″, 0.04″, 0.055″, 0.084″, 0.09″.

In some embodiments, the side opening 110 comprises a size (or diameter) of from about 0.015″ to about 0.1″. For example, 0.015″, 0.02″, 0.036″, 0.04″, 0.055″, 0.084″, 0.09″.

In some embodiments, the side opening 110 is located at a distance from the distal opening 108 of from about 0.5 mm to about 10 mm. Optionally from about 0.3 mm to about 20 mm. Optionally from about 0.1 mm to about 50 mm.

In some embodiments, the external tubular body 102 is made of one or more of nylon, Pebax, polyorithen, metal, PTFE and any combination thereof. In some embodiments, the external tubular body is a braided/coiled shaft.

In some embodiments, the external tubular body 102 comprises a length of from about 90 cm to about 180 cm. Optionally from about 80 cm to about 200 cm. Optionally from about 50 cm to about 250 cm. In some embodiments, the external tubular body 102 comprises a size that fits through a 5 F or 6 F sheath.

In some embodiments, the redirection microcatheter 100 comprises an internal tubular body 112 comprising a proximal end 114 and a distal end 116. In some embodiments, the proximal end 114 of the internal tubular body 112 is connected to the actuation handle (not shown). In some embodiments, the distal end 116 of the internal tubular body 112 extends distally up to the vicinity of the location of the side opening 110. In some embodiments, the internal tubular body 112 is made of a flexible material. In some embodiments, at least a part of the internal tubular body 112 is made of a flexible material. In some embodiments, at least a distal part of the internal tubular body 112 is made of a flexible material.

In some embodiments, the flexible part of the internal tubular body 112 comprises a hardness of from about 20 A Shore to about 60 A Shore; optionally from about 10 A Shore to about 75 A Shore; optionally from about 0 A Shore to about 100 A Shore; for example 70 A Shore, 50 A shore, 25 A Shore. In some embodiments, the flexible part of the internal tubular body 112 comprises a hardness of from about 20 D Shore to about 60 D Shore; optionally from about 10 D Shore to about 75 D Shore; optionally from about 0 D Shore to about 100 D Shore; for example 55 D Shore, 40 D shore, 2 5D Shore.

In some embodiments, the internal tubular body 112 is made of one or more of nylon, Pebax, polyorithen, metal, PTFE and any combination thereof. In some embodiments, the external tubular body is a braided/coiled shaft.

In some embodiments, the internal tubular body 112 comprises a length of from about 100 mm to about 1800 mm. Optionally from about 50 mm to about 2000 mm. Optionally from about 25 mm to about 2500 mm. In some embodiments, the internal tubular body 112 comprises a length of about of that of the external tubular body 102.

In some embodiments, the distal end 116 of the internal tubular body 112 extends from the proximal end of the device until a distance of from about 0.1 cm to about 1.5 cm from the distal end 106 of the external tubular body 102.

In some embodiments, the internal tubular body 112 is configured to be extracted from the redirection microcatheter 100 to allow insertion of other instruments within the redirection microcatheter 100. In some embodiments, the internal tubular body 112 is configured to allow insertion of other instruments within itself without the need to extract the internal tubular body 112.

In some embodiments, the redirection microcatheter 100 comprises an actuator 118. In some embodiments, the actuator 118 is a wire. In some embodiments, the actuator 118 is a push-pull wire. In some embodiments, the actuator 118 is made of one or more of stainless steel, nitinol, cobalt chromium, nylon, peek and Dynema.

In some embodiments, the actuator 118 comprises a proximal end 120 and a distal end 122. In some embodiments, the proximal end 120 of the actuator 118 extends to the inside of the actuation handle (not shown), to an actuation mechanism, which will be further explained below. In some embodiments, the distal end 122 of the actuator 118 is connected to a holder 124 (see below) located adjacent to the distal end 116 of the internal tubular body 112.

In some embodiments, the redirection microcatheter 100 comprises a holder 124. In some embodiments, the holder 124 is connected to the internal tubular body 112 in a location adjacent to the distal end 116 of the internal tubular body 112. In some embodiments, the holder 124 is connected to at least a part of the flexible part of the internal tubular body 112. In some embodiments, the location of the holder 124 is from about 1 mm to about 20 mm from the distal end 116 of the internal tubular body 112. Optionally from about 0.5 mm to about 40 mm. Optionally from about 0.1 mm to about 60 mm.

In some embodiments, the holder 124 is a ring or a band. In some embodiments, the holder 124 is arranged around the internal tubular body 112. In some embodiments, the holder 124 is a grove on the surface of the internal tubular body 112 and the distal end 122 of the actuator is positioned within the groove.

In some embodiments, the holder 124 is made of one or more of stainless steel, nitinol, cobalt chromium, nylon, peek, platinum iridium and gold.

In some embodiments, actuation of the actuator 118 causes a part of the distal end 116 of the internal tubular body 112 to translate from a first position to a second position. In some embodiments, the first position is a resting position in which the part of the distal end 116 of the internal tubular body 112 is not actuated and the distal end 116 of the internal tubular body 112 is aligned with a longitudinal axis 126 of the redirection microcatheter 100, as shown for example in FIG. 1a. In some embodiments, the second position is an actuated position in which the part of the distal end 116 of the internal tubular body 112 is actuated and translated from the longitudinal axis 126 to face and/or be directed (see arrow 128) towards the side opening 110, as shown for example in FIG. 1b. An exemplary use of a push-pull wire will be used as an exemplary actuator for the following explanations. It should be understood that this is done in order to allow a person having skills in the art to understand the invention and should not restrict the invention in any way. In some embodiments, the push-pull wire actuates the internal tubular body 112 from the first position to the second position (or from the un-actuated position to the actuated position). In some embodiments, the push-pull wire actuates the internal tubular body 112 from the second position to the first position (or from the actuated position to the un-actuated position). In some embodiments, the internal tubular body 112 natural state is in the rest position (un-actuated position) and, during actuation, the internal tubular body 112 will “want” to return to the rest position. Therefore, in some embodiments, actuation of the internal tubular body 112 comprises actively translating the part of the distal end 116 of the internal tubular body 112 from a resting position to an actuated position by pulling the push-pull wire and holding the push-pull wire tense until actuation is no longer required. Then, in some embodiments, releasing and/or reducing the tension of the push-pull wire allows the internal tubular body 112 to return to the natural resting position.

In some embodiments, the translation during the actuation is from about 1 degree to about 90 degrees in relation to the longitudinal axis of the longitudinal axis 126 of the redirection microcatheter 100. Optionally, from about 1 degree to about 150 degrees. Optionally from about 1 degree to about 170 degrees.

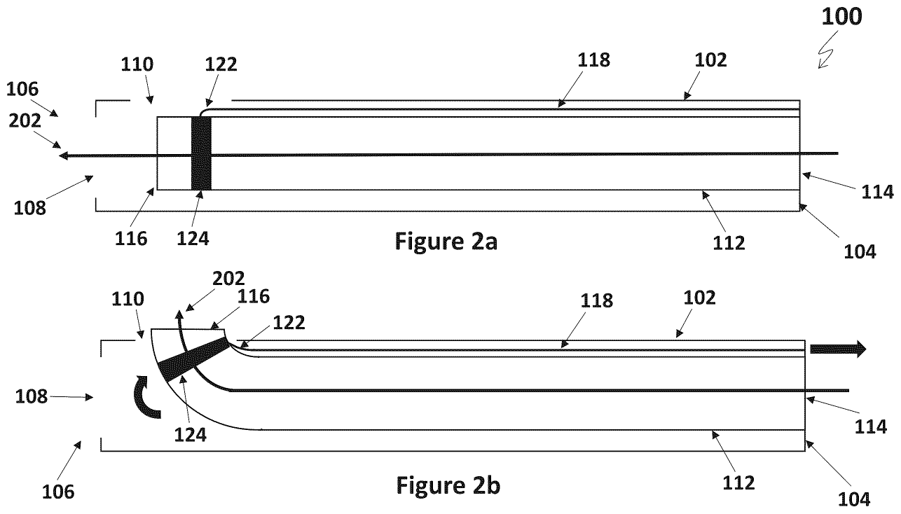

Referring now to FIGS. 1c-1e, showing schematic representations of an exemplary redirection microcatheter 100 comprising a reinforced area, according to some embodiments of the invention. In some embodiments, in the area around the side opening 110, the redirection microcatheter 100 comprises a reinforced area 130. In some embodiments, the reinforced area comprises one or more metallic elements that confer a certain level of rigidity to the reinforced area, which is higher than a level of rigidity of areas without the metallic elements. For example, the external tubular body 102 may be made of a bendable material (for example PEBAX), and the reinforced area may comprise a metallic braid imbedded within the external tubular body 102 and extending over the area around the side opening 110, as schematically shown, for example, in FIG. 1e. In some embodiments, the metallic elements comprise any suitable form, for example, it can be a metallic braid, a metallic scaffold, metallic rods, metallic rings, etc. In some embodiments, the metallic elements are located on parts the external tubular body 102, meaning that not all the area around the side opening 110 is reinforced. For example, reinforced areas can be opposite to the side opening 110, at two location near the side opening 110, etc. In some embodiments, the reinforced area 130 does not obstruct the side opening 110. In some embodiments, a potential advantage of having a reinforced area 130 around the side opening 110, is that it potentially avoid unwanted bending of the external tubular body 102 due to the forces applied when actuating the internal tubular body 112 for redirecting a device located within the internal tubular body 112. In some embodiments, the reinforced area 130 is also present in the embodiments as disclosed in FIGS. 2a-b, 3a-b and 4a-b.

Referring now to FIGS. 2a-2b, showing schematic representations of an exemplary redirection microcatheter 100 with a guidewire 202, according to some embodiments of the invention. In some embodiments, the internal tubular body 112 allows the insertion of a guidewire 202 within, which is used, for example, for directing the redirection microcatheter 100 to the desired location. In some embodiments, the redirection microcatheter 100 is configured to allow the insertion and extraction of the guidewire 202 inside the internal tubular body 112. In some embodiments, a distal end of the guidewire 202 can exit the internal tubular body 112 and the distal end 106 of the redirection microcatheter 100 using the distal opening 108, as shown for example in FIG. 2a. This will be referred hereinafter as “distal exiting” of the guide wire. In some embodiments, this is allowed when the internal tubular body 112 is in a resting position. In some embodiments, when actuated, the distal end of the guidewire 202 can exit the internal tubular body 112 and the redirection microcatheter 100 using the side opening 110, as shown for example in FIG. 2b. This will be referred hereinafter as “side exiting” of the guide wire. In some embodiments, the passage of the guidewire 202 from distal exiting to side exiting, and vice versa, requires the insertion of the guidewire 202 into the internal tubular body 112 before the actuation of the internal tubular body 112 from one configuration (actuated or non-actuated) to another.

In some embodiments, the guidewire 202 can be a 0.035 mm guidewire or a 0.018 mm guidewire or a 0.014 mm guidewire or a 0.009 guidewire.

### Exemplary Multi-Lumen Redirection Microcatheter

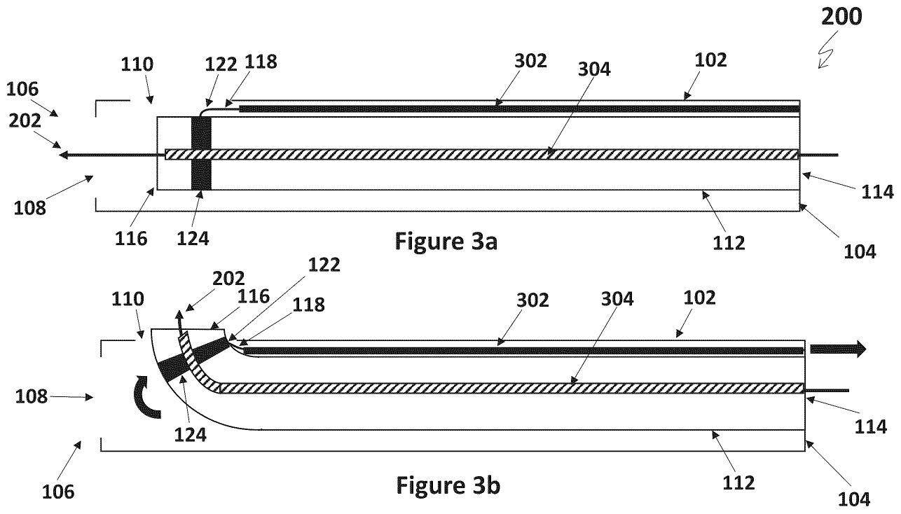

Referring now to FIGS. 3a-b, showing an exemplary multi-lumen redirection catheter 200, according to some embodiments of the invention. In some embodiments, one or more of the components disclosed above are incorporated within a dedicated lumen, optionally of a dedicated elongated member, for example, a tube.

For example, in some embodiments, the actuator 118 is incorporated within a dedicated tube 302 extending from the proximal end 104 of the external tubular body 102 until a location adjacent to the holder 124. In some embodiments, the dedicated tube 302 extends in a zone within the external tubular body 102 and outside the internal tubular body 112.

Another example, in some embodiments, the guidewire 202 is incorporated within a dedicated tube 304 extending from the proximal end 104 of the internal tubular body 112 until a location adjacent to the distal end 116 of the internal tubular body 112. In some embodiments, the dedicated tube 304 extends in a zone within the internal tubular body 112.

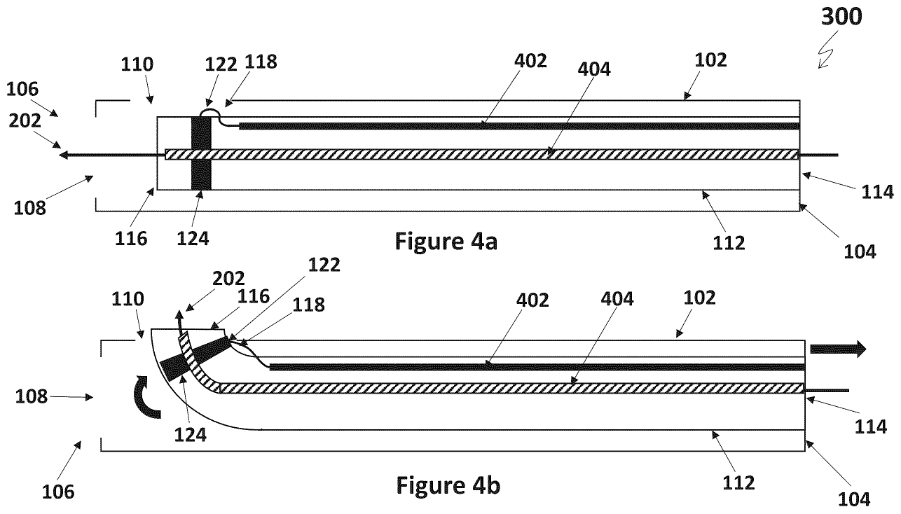

Referring now to FIGS. 4a-b, showing another exemplary multi-lumen redirection catheter 300, according to some embodiments of the invention. In some embodiments, the actuator 118 is incorporated within a dedicated tube 402 extending from the proximal end 104 of the external tubular body 102 until a location adjacent to the holder 124. In some embodiments, the dedicated tube 402 extends in a zone within the internal tubular body 112. Additionally, in some embodiments, the guidewire 202 is incorporated within a dedicated tube 404 extending from the proximal end 104 of the internal tubular body 112 until a location adjacent to the distal end 116 of the internal tubular body 112. In some embodiments, the dedicated tube 404 extends in a zone within the internal tubular body 112.

### Exemplary Handle

In some embodiments, the device comprises a handle at a proximal end. In some embodiments, the handle comprises dedicated mechanisms to actuate one or more mechanisms of the catheter, as known in the art. In some embodiments, the handle comprises an actuator to which the push-pull wire is connected. In some embodiments, a user actuates the actuator causing the pulling of the push-pull wire, therefore causing the internal tubular body to translate towards the one or more side openings. In some embodiments, the user actuates the actuator again to release and/or push the push-pull wire, thereby causing a translation of the internal tubular body to its original position facing the distal opening of the device. In some embodiments, the actuator comprises two modalities: a free actuation mode, in which the user actuates the actuator freely from one position to another; and a semi-locked actuation mode, in which actuation of the actuator is done by steps, for example, each time the user actuated the actuator, the internal tubular body is translated a certain quantity, for example 10 degrees. In some embodiments, this means, for example, that for the internal tubular body to move to a 90 degrees angle, the user needs to actuate the actuator nine times. In some embodiments, in the semi-locked actuation mode, after each actuation step, the actuator stays at the last actuation position. In some embodiments, in the free actuation mode, the user holds the tension of the push-pull wire by holding the actuator and releasing the actuator will allow the internal tubular body to return to the resting un-actuated position.

### Exemplary Methods

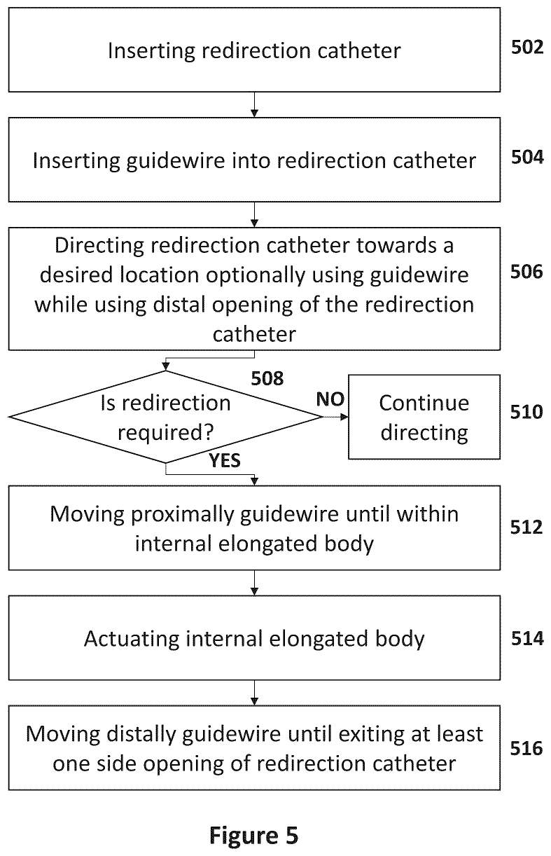

Referring now to FIG. 5, showing a flowchart of an exemplary method, according to some embodiments of the invention. In some embodiments, a user inserts the redirection catheter into a patient 502. In some embodiments, the user inserts the guidewire into the redirection catheter 504. In some embodiments, the guidewire is already inside the redirection catheter when the redirection catheter is inserted into the patient. In some embodiments, the user directs the redirection catheter towards a desired location using known methods 506, optionally using the guidewire to assist with the direction. In some embodiments, at this point the guidewire exists the redirection catheter using a distal opening in the redirection catheter. In some embodiments, at some point of the directing, the user assesses whether redirection of the redirecting catheter is required 508. In some embodiments, if the answer is no, then the user continues with the directing of the redirection catheter 510. In some embodiments, if the answer is yes, then the user moves the guidewire proximally until the distal end of the guidewire reaches the internal tubular body 512. In some embodiments, once the distal end of the guidewire is within the internal tubular body, the user actuates the internal tubular body 514 to cause a translation of the distal end of the internal tubular body from a first position to a second position, where the first position is a position where the internal tubular body follows a longitudinal axis of the redirection catheter and the second position is a position where the distal end of the internal tubular body is redirected towards at least one side opening in the redirection catheter. In some embodiments, then the user moves distally the guidewire 516 to cause the guidewire to exit from at least one side opening in the redirection catheter. In some embodiments, once the guidewire is in position, the user can push the redirection catheter to enter in the right location. In some embodiments, once in the right location, the user retracts the guidewire, returns the internal tubular body into the resting position, and continues directing the device to the desired location.

### Exemplary Markings

In some embodiments, an exemplary redirection catheter comprises one or more markings that assist the user in identifying the direction to which the side opening 110 is facing. For example, radiopaque markers are positioned adjacent to the side opening 110, which can be seen using known visualization techniques. Another example, the side opening 110 is marked, for example with a half or whole circle, surrounding the opening itself. In some embodiments, radiopaque markings are added on both the surface of the external tubular body 102 as dots (for example) and around the opening itself.

In some embodiments, elements of the redirection catheter can be made of or added with radiopaque materials. For example, one or more of the actuator 118 of the redirection mechanism, the holder 124 and a dedicated ring located at the most distal end of the external tubular body 102, can be made of or added with radiopaque materials. In some embodiments, a potential advantage of marking the actuator 118 is that it will provide a “line” showing directly the direction of the opening.

In some embodiments, additionally or alternatively, the redirection catheter comprises markings at the handle, which show the user the direction which the side opening 110 is facing.

In some embodiments, additionally or alternatively, the redirection catheter comprises an actuation element on the handle, which, once actuated, performs the redirection action. In some embodiments, the location of the actuation element on the handle shows the user the direction to which the opening is facing since it is located on a same side as the opening.

### Exemplary Optional Stabilizer

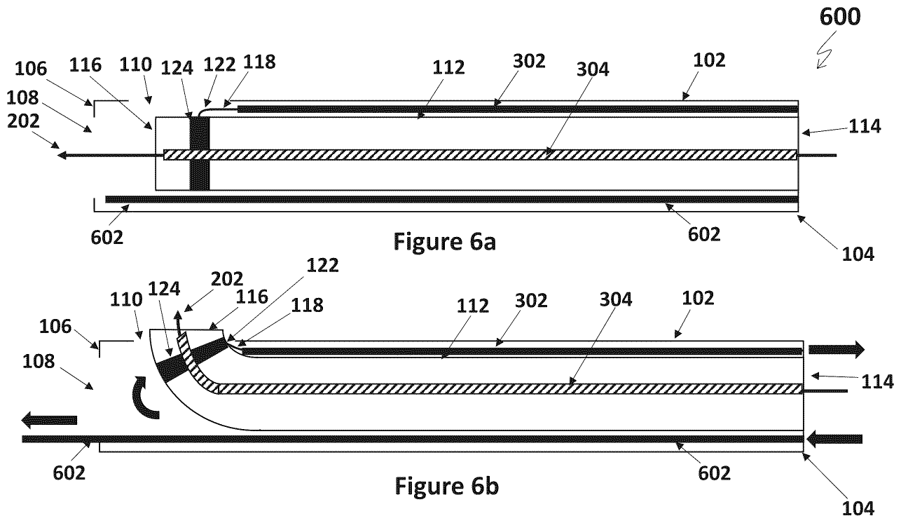

Referring now to FIGS. 6a-b, showing an exemplary optional stabilizer, according to some embodiments of the invention. In some embodiments, an exemplary redirection catheter 600 optionally comprises a stabilizer 602. In some embodiments, the stabilizer 602 comprises a wire extending from the most proximal end 104 to the most distal end 106 of the external tubular body 102. In some embodiments, the stabilizer 602 is positioned between the external tubular body 102 and the internal tubular body 112. In some embodiments, the stabilizer 602 either comprises its own dedicated tube (not shown) or just runs within the lumen of the external tubular body 102, same as the internal tubular body 112.

In some embodiments, the stabilizer 602 is a metal wire. In some embodiments, the stabilizer 602 comprises a diameter of from about 150 micrometer to about 0.5 mm, In some embodiments, the stabilizer 602 comprises a strength that is high enough to stop the external tube from bending when the redirection actuator is being actuated, but is low enough so as not to affect the flexibility of the external tube, and therefore of the redirection catheter.

In some embodiments, during the operation of the redirection catheter 600, when the user wants to use the most distal opening 108 of the external tubular body 102 as the exit for the guidewire 202—distal exiting of the guidewire 202, the stabilizer 602 is kept within the lumen of the external tubular body 102—which is the inactive state of the stabilizing element, as schematically shown in FIG. 6a.

In some embodiments, as mentioned above, during the operation of the redirection catheter 600, when the user wants to redirect the guidewire 202 to exit through side opening 110—side exiting, the user needs to cause the bending of the internal tubular body 112 towards the side opening 110 by actuating the actuator 118. In some embodiments, before or during the act of actuating the redirection mechanism, the user “activates” the stabilizing mechanism by causing the stabilizer 602 to exit the lumen of the external tubular body 102 through the most distal opening 108 of the external tubular body 102—which is the active state of the stabilizing element, as schematically shown in FIG. 6b. In some embodiments, activation of the stabilizer 602 is by pushing it forwards in the direction of the distal end of the redirection catheter, while inactivating the stabilizer 602 is by pulling it backwards in the direction of the proximal end of the redirection catheter.

In some embodiments, a potential advantage of having a stabilizer 602 is that it potentially avoids the bending of the external tubular body 102 during the actuation of the redirection mechanism, meaning during the bending of the internal tubular body 112 during the redirection act.

### Exemplary Methods with Stabilizing Element

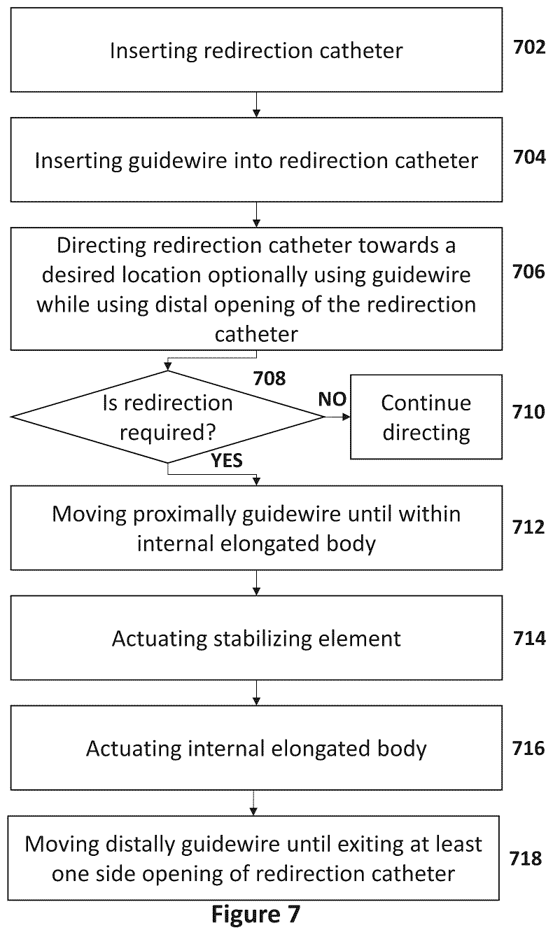

Referring now to FIG. 7, showing a flowchart of an exemplary method using the stabilizer, according to some embodiments of the invention. In some embodiments, a user inserts the redirection catheter into a patient 702. In some embodiments, the user inserts the guidewire into the redirection catheter 704. In some embodiments, the guidewire is already inside the redirection catheter when the redirection catheter is inserted into the patient. In some embodiments, the user directs the redirection catheter towards a desired location using known methods 706, optionally using the guidewire to assist with the direction. In some embodiments, at this point the guidewire exists the redirection catheter using a distal opening in the redirection catheter. In some embodiments, at some point of the directing, the user assesses whether redirection of the redirecting catheter is required 708. In some embodiments, if the answer is no, then the user continues with the directing of the redirection catheter 710. In some embodiments, if the answer is yes, then the user moves the guidewire proximally until the distal end of the guidewire reaches the internal tubular body 712. In some embodiments, once the distal end of the guidewire is within the internal tubular body, the user actuates the stabilizer by causing it to exit through the distal end of the external tubular body 714. In some embodiments, after activation of the stabilizer, the user actuates the internal tubular body 716 to cause a translation of the distal end of the internal tubular body from a first position to a second position, where the first position is a position where the internal tubular body follows a longitudinal axis of the redirection catheter and the second position is a position where the distal end of the internal tubular body is redirected towards at least one side opening in the redirection catheter. In some embodiments, then the user moves distally the guidewire 718 to cause the guidewire to exit from at least one side opening in the redirection catheter. In some embodiments, once the guidewire is in position, the user can push the redirection catheter to enter in the right location. In some embodiments, once in the right location, the user retracts the guidewire, returns the internal tubular body into the resting position, and continues directing the device to the desired location.

As used herein with reference to quantity or value, the term “about” means “within +20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements. Various embodiments and aspects of the present invention as delineated hereinabove and as claimed in the claims section below find experimental/calculated support in the following exemplary embodiments.

### EXAMPLARY EMBODIMENTS

Reference is now made to the following exemplary embodiments, which together with the above descriptions illustrate some embodiments of the invention in a non-limiting fashion.

In some embodiments, the device is used as a re-entry microcatheter for chronic total occlusions (CTO) in one or more of the coronary arteries, the peripheral arteries, cerebrovascular vessels, aortic arch vessels, intra-abdominal vessels and peripheral arteries (for example, leg arteries, including superficial femoral, popliteal, anterior tibial, etc.).

In some embodiments, the device is used as a redirection catheter, as disclosed above.

In some embodiments, the common principle for both procedures is to facilitate guidewire advancement across different angulations into branch vessels or side vessels to enable a treatment such as a balloon angioplasty, a stent, an embolic device such as coils andor delivery of a medication (for example: chemotherapy). In some embodiments, due to a sharp angulation or the presence of some obstructing material such as stent strut, the guidewire is not able to enter the desired area. In some embodiments, the guidewire must first cross into the branch or side vessel in order to deliver the balloon catheter, the stent catheter or the microcatheter (or drug therapy, embolic coils, etc.).

For example:
a. coronary example:
   i. A first example, angulated large vessels, an angulated origin of the left anterior descending artery (LAD) or the circumflex artery (Cx) off the left main coronary artery and there is a lesion distally in the LAD or the Cx.
   ii. A second example, angulated branch vessels, an angulated diagonal branch of the LAD or an angulated obtuse marginal branch of the circumflex or the distal branches of the right coronary artery (RCA).
   iii. A third example, after placing a stent, the operator may want to access a diagonal side branch of a stented LAD or an obtuse marginal branch of a stented Cx. It happens that guidewires cannot advance between the struts of the stent into the branch vessel.
   iv. A fourth example, trying to advance guidewires into coronary arteries or bypass grafts that have difficult to access origins (also called ostia) and the guiding catheter is unable to engage the ostium.

Other examples include: angulated lesions where guidewire not advancing in desired path, branch vessels through stent struts, proximal cap of CTOs where initial penetration is not in correct place and retrograde in bypass grafts back into native vessels.
b. non coronary arteries example: accessing branches of the aortic arch (e.g. left subclavian, left carotid, brachiocephalic) due to an extreme angle and an intervention is required in the branch vessel or a branch of the branch vessel. For example, if doing a right radial approach for a patient post bypass surgery and trying to do a left internal mammary artery (LIMA) injection to check the patency of the bypass, it is difficult to access the left subclavian artery from the right radial approach due to the angulation of the left subclavian artery from this approach. Although you could potentially do the case from a femoral artery approach, this may be very undesirable for many reasons (very heavy patient, severe peripheral vascular disease, etc.).

Other examples include: Peripherals (including abdominals and neck/intracerebral vessels), angulated branch vessels and proximal cap of CTOs where initial penetration is not in correct place.

Additionally, other examples for 0.035 mm redirection microcatheter includes: treatments in the lower extremity, abdomen, arch vessels, (e.g. left subclavian, left carotid, brachiocephalic, including subclavian (LIMA) from right radial approach) and proximal cap of CTOs where initial penetration is not in correct place. For example, right radial approach for a patient post bypass surgery is extremely difficult to access left internal mammary artery (LIMA) injection to check the patency of the LIMA, due to angulation of the left subclavian artery off the aorta. Although you could potentially do the case from a femoral artery approach, this may be very undesirable for many reasons (very heavy patient, severe peripheral vascular disease, etc.).

Additional examples are accessing specific arteries in the abdomen (e.g. liver) for chemotherapy delivery, or endometrial artery for treatment of leiomyomas (fibroids). Sometimes in an angulated take-off, even though the guidewire may advance, the microcatheter will not follow and prolapse. There are also cases in the peripheral arteries where access to a superficial femoral artery may be challenging and the guidewire only will engage the profunda femoral artery.
c. Additional examples are: accessing specific arteries in the abdomen (e.g. liver) for chemotherapy delivery, or endometrial artery for treatment of leiomyomas (fibroids). Additional uses for this invention can be one or more of LIMA angiograms from right radial approach, delivery of intra-abdominal chemotherapy in tortuous arteries (hepatic cell carcinoma, liver metastatic disease (colorectal, pancreatic, breast), uterine fibroid embolization), coronary structural procedures including access to coronary sinus, coronary artery access after TAVI, paravalvular leak crossing), peripheral artery disease interventions with lower limb tortuosity (especially below knees), renal artery access, chronic total occlusion crossing (coronary arteries and peripheral arteries), creating arterial- venous anastomosis, crossing of atrial septal defects, patent foramen ovale and ventricular septal defects (VSD).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A redirection microcatheter, comprising:
a. an external tubular body comprising a first proximal end and a first distal end; said first distal end comprising a distal end opening and at least one side opening;
b. an internal tubular body sized and shaped to be inserted within said external tubular body; said internal tubular body comprising a second proximal end and a second distal end; said second distal end located adjacent to said at least one side opening;
c. at least one actuator for translating said second distal end of said internal tubular body from an un-actuated position where said second distal end of said internal tubular body faces said distal opening to an actuated position where said second distal end of said internal tubular body faces said one or more side openings in said external tubular body.

2. The redirection catheter according to claim 1, wherein said actuator comprises at least one holder coupled to a location adjacent to said second distal end of said internal tubular body, and at least one wire coupled to said at least one holder; and wherein said actuator is made of one or more of stainless steel, nitinol, cobalt chromium, nylon, peek and Dynema.

3. The redirection catheter according to claim 1, wherein said translating comprises at least one of:
a. bending said second distal end of said internal tubular body an angle of from about 1 degree to about 150 degrees;
b. bending said second distal end of said internal tubular body an angle of about 90 degrees.

4. (canceled)

5. The redirection catheter according to claim 1, wherein said at least one side opening is at a distance of from about 0.5 mm to about 10 mm from said distal opening and wherein said at least one side opening comprises a size of from about 0.015″ to about 0.1″.

6. The redirection catheter according to claim 1, wherein said distal opening comprises a size of from about 0.015″ to about 0.1″.

7. (canceled)

8. The redirection catheter according to claim 1, wherein said external tubular body is characterized by one or more of:
a. being made of one or more of nylon, Pebax, polyorithen, metal, PTFE and any combination thereof;
b. is a braided/coiled shaft;
c. comprises a length of from about 90 cm to about 180 cm;
d. comprises a size that fits through a 5 F or 6 F sheath;
e. comprises a reinforced area located adjacent and/or around to said one or more side openings;
f. is a braided/coiled shaft.

9-12. (canceled)

13. The redirection catheter according to claim 8, wherein said reinforced area comprises one or more metallic elements configured to increase a level of rigidity in said reinforced area.

14. The redirection catheter according to claim 1, wherein said internal tubular body is characterized by one or more of:
a. being made of a flexible material;
b. being made at least partially of a flexible material;
c. having a distal end comprised at least partially of a flexible material;
d. being made of one or more of nylon, Pebax, polyorithen, metal, PTFE and any combination thereof;
e. having a length of from about 100 mm to about 1800 mm;
f. having a length of about of that of said external tubular body;
g. being configured to be extracted from said external tubular body thereby allowing insertion of other instruments within said external tubular body;
h. being configured to allow insertion of other instruments within itself without the need to be extracted from said external tubular body;
i. being sized and shaped to allow the insertion of at least one guidewire within;
j. having a third internal lumen in which said at least one guidewire is inserted.

15-16. (canceled)

17. The redirection catheter according to claim 14, wherein a part of said internal tubular body which is made of a flexible material comprises a hardness of from about 20 D Shore to about 60 D Shore.

18-21. (canceled)

22. The redirection catheter according to claim 1, wherein said second distal end extends until a distance of from about 0.1 cm to about 1.5 cm from said first distal end.

23-25. (canceled)

26. The redirection catheter according to claim 2, wherein said at least one holder is characterized by one or more of:
a. being a ring;
b. being positioned on a flexible part of said internal tubular body;
c. being positioned at a distance of from about 1 mm to about 20 mm from said second distal end;
d. being arranged around said internal tubular body;
e. being a groove on a surface of said internal tubular body;
f. being made of one or more of stainless steel, nitinol, cobalt chromium, nylon, peek, platinum iridium and gold.

27-32. (canceled)

33. The redirection catheter according to claim 1, wherein said external tubular body comprises a first internal lumen in which said at least one actuator extends.

34-35. (canceled)

36. The redirection catheter according to claim 1, further comprising at least one handle located proximally in said redirection catheter configured to receive one or more of said first proximal end of said external tubular body, said second proximal end of said internal tubular body and a third proximal end of said actuator.

37. The redirection catheter according to claim 1, further comprising at least one marking configured to indicate a direction to which said one or more side openings are facing; and wherein said at least one marking is characterized by one or more of:
a. being a radiopaque marking;
b. being located in one or more of the following locations: on the surface of said external tubular body, on the surface of said first distal end of said external tubular body, adjacent to said one or more side openings, on said at least one actuator and on a at least one holder.

38-39. (canceled)

40. The redirection catheter according to claim 1, further comprising a stabilizer configured to avoid bending of said first distal end when said at least one actuator is being actuated; and wherein said stabilizer is characterized by one or more of:
a. extending from said first proximal end to said first distal end;
b. extending within a lumen of said external tubular body;
c. extending extends between said external tubular body and said internal tubular body;
d. extending extends within a lumen of said external tubular body but outside of said internal tubular body;
e. being configured to extend beyond said distal end opening when actuated.

41-45. (canceled)

46. A method of actuating a redirection catheter according to claim 1 while using a guidewire, the method comprising:
a. retracting said guidewire from said distal opening of said redirection catheter;
b. actuating said internal tubular body thereby translating said second distal end of said internal tubular body from an un-actuated position where said second distal end of said internal tubular body faces said distal opening to an actuated position where said second distal end of said internal tubular body faces said one or more side openings in said external tubular body;
c. moving distally said guidewire to cause said guidewire to exit from said at least one side opening in said redirection catheter.

47. The method according to claim 46, further comprising inserting said redirection catheter into a patient.

48. The method according to claim 46, further comprising directing said redirection catheter towards a desired location using said guidewire to assist with said directing.

49. The method according to claim 46, wherein said inserting further comprises inserting said guidewire into said redirection catheter.

50. The method according to claim 46, wherein said redirection catheter already comprises said guidewire inserted therein.
